(19) United States
(12) Patent Application Publication
(10) Pub. No.: US 2008/0161205 A1
(43) Pub. Date: Jul. 3, 2008

(54) METHOD OF TWO-DIMENSIONALLY ARRAYING FERRITIN ON SUBSTRATE

(76) Inventors: Takuro Matsui, Nara (JP); Nozomu Matsukawa, Nara (JP)

Correspondence Address: MCDERMOTT WILL & EMERY LLP, 600 13TH STREET, NW, WASHINGTON, DC, 20005-3096, US

(21) Appl. No.: 11/952,609

(22) Filed: Dec. 7, 2007

(30) Foreign Application Priority Data
Dec. 7, 2006 (JP) 2006-330767

(51) Int. Cl. C40B 50/18

(52) U.S. Cl. 506/32

(57) ABSTRACT

The present invention provides a novel method of two-dimensionally arraying ferritin on a substrate, which obviates the need for a metal ion for achieving linking between two adjacent ferritin. The present invention provides a method of two-dimensionally arraying ferritin on a substrate, wherein the ferritin has an amino acid sequence set out in SEQ ID NO: 1 on the outer peripheral surface; the surface of the substrate is hydrophilic; and the method includes a development step of developing a solution that contains a solvent, the ferritin, and 2 mM to 100 mM ammonium acetate on the substrate, and a removal step of removing the solvent from the solution developed on the substrate.

### BACKGROUND OF THE INVENTION

1. Field of the Invention

The present invention relates to a method of two-dimensionally arraying ferritin on a substrate, and more specifically, relates to a method which obviates the need for a metal ion that permits linking between two adjacent ferritin.

2. Description of Related Art

Ferritin is a spherical protein that includes a metal compound therein which is typified by iron oxide. When it does not include any metal compound therein but has a hollow space, it is referred to as “apoferritin”.

Quantum dots of a metal that is two-dimensionally arrayed on a substrate can be readily obtained by two-dimensionally arraying ferritin on the substrate followed by removing the ferritin by heat, and reducing metal oxide if necessary.

Accordingly, two-dimensionally arraying of ferritin on a substrate as shown in FIG. 1 has been attempted so far (for example, see pamphlet of International Publication No. 03/040025 (hereinafter, referred to as Patent Document 1)).

### SUMMARY OF THE INVENTION

According to the method disclosed in Patent Document 1, as shown in FIG. 25, crosslinking between two adjacent ferritin is effected via a bivalent metal ion (cadmium ion in FIG. 25).

After removing ferritin by heat, this bivalent metal ion remains on the substrate as an impurity.

The impurity is supposed to migrate on the substrate in the form of an ion, therefore, an unexpected interface state may be generated due to such an impurity in the quantum dots composed of a two-dimensional array of a metal on a substrate.

As a consequence, this impurity adversely affects the quantum dots.

According to the present invention, a novel method of two-dimensionally arraying ferritin on a substrate, which is not accompanied by such an adverse effect, that is, a method which obviates the need for a metal ion for achieving linking between two adjacent ferritin is provided.

In order to solve the problems described above, the present invention involves a method of two-dimensionally arraying ferritin on a substrate, wherein the ferritin has an amino acid sequence set out in SEQ ID NO: 1 on the outer peripheral surface; the surface of the substrate is hydrophilic; and the method includes a development step of developing a solution that contains a solvent, the ferritin, and 2 mM to 100 mM ammonium acetate on the substrate, and a removal step of removing the solvent from the solution developed on the substrate.

### BRIEF DESCRIPTION OF THE DRAWINGS

FIG. 1 shows a schematic view illustrating a state in which multiple ferritin 15 molecules forms a two-dimensional array on substrate 11.

FIG. 2 shows a cross-sectional view illustrating a three-dimensional array.

FIG. 3 shows a photograph illustrating the appearance of a two-dimensional array of ferritin obtained in Example 1.

FIG. 4 shows a photograph illustrating the appearance of a two-dimensional array of ferritin obtained in Example 2.

FIG. 5 shows a photograph illustrating the appearance of a two-dimensional array of ferritin obtained in Example 3.

FIG. 6 shows a photograph illustrating the appearance of a two-dimensional array of ferritin obtained in Example 4.

FIG. 7 shows a photograph illustrating the appearance of a two-dimensional array of ferritin obtained in Example 5.

FIG. 8 shows a photograph illustrating the appearance of a two-dimensional array of ferritin obtained in Example 6.

FIG. 9 shows a photograph illustrating the appearance of a two-dimensional array of ferritin obtained in Example 7.

FIG. 10 shows a photograph illustrating the appearance of a two-dimensional array of ferritin obtained in Example 8.

FIG. 11 shows a photograph illustrating the appearance of a two-dimensional array of ferritin obtained in Example 9.

FIG. 12 shows a photograph illustrating the appearance of a two-dimensional array of ferritin obtained in Example 10.

FIG. 13 shows a photograph illustrating the appearance of a two-dimensional array of ferritin obtained in Example 11.

FIG. 14 shows a photograph illustrating the appearance of a two-dimensional array of ferritin obtained in Example 12.

FIG. 15 shows a photograph illustrating the appearance of ferritin on the substrate obtained in Comparative Example 1.

FIG. 16 shows a photograph illustrating the appearance of ferritin on the substrate obtained in Comparative Example 2.

FIG. 17 shows a photograph illustrating the appearance of ferritin on the substrate obtained in Comparative Example 3.

FIG. 18 shows a photograph illustrating the appearance of ferritin on the substrate obtained in Comparative Example 4.

FIG. 19 shows a photograph illustrating the appearance of ferritin on the substrate obtained in Comparative Example 5.

FIG. 20 shows a photograph illustrating the appearance of ferritin on the substrate obtained in Comparative Example 6.

FIG. 21 shows a photograph illustrating the appearance of ferritin on the substrate obtained in Comparative Example 7.

FIG. 22 shows a photograph illustrating the appearance of ferritin on the substrate obtained in Comparative Example 8.

FIG. 23 shows a photograph illustrating the appearance of ferritin on the substrate obtained in Comparative Example 9.

FIG. 24 shows a photograph illustrating the appearance of ferritin on the substrate obtained in Comparative Example 10.

FIG. 25 shows a schematic view illustrating a state in which crosslinking between two adjacent ferritin is effected via a bivalent metal ion (cadmium ion in FIG. 25), as shown in FIG. 8 of Patent Document 1.

### DETAILED DESCRIPTION OF PREFERRED EMBODIMENTS

The present invention will be explained below in more detail.

Ferritin used in the present invention has an amino acid sequence of DYFSSPYYEQLF (hereinafter, SEQ ID NO: 1) on the outer peripheral surface. This amino acid sequence is disclosed in Japanese Unexamined Patent Application Publication No. 2004-121154 with designation of “pNHD12-5-2”. By way of example, ferritin used in the present invention is a protein set out in SEQ ID NO: 2. This protein has 187 residues, including an amino acid sequence having 174 residues of ferritin derived from horse, to which an amino acid sequence having 13 residues which includes methionine corresponding to an initiation codon and an amino acid sequence set out in SEQ ID NO: 1 was added at the amino terminal.

In Experimental Example described later, ferritin used in the present invention is denoted as “CNHB-Fer0”. In the case of apoferritin, it is denoted as “apoCNHB-Fer0”. The aforementioned ferritin having 174 residues derived from horse is denoted as “Fer0”.

General ferritin does not have the amino acid sequence setout in SEQ ID NO: 1. As will be understood also from Comparative Examples described later, a two-dimensional array cannot be formed on a substrate even though ferritin not having the amino acid sequence set out in SEQ ID NO: 1 is used including general ferritin.

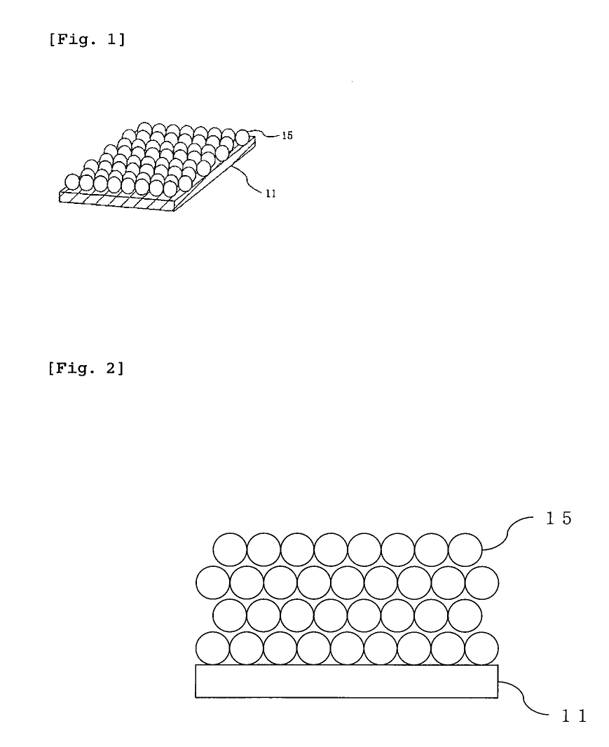

The term “two-dimensional array” as used herein means, as shown in a schematic view in FIG. 1, an array in which multiple ferritin 15 molecules are regularly arranged on a substrate 11 as viewed in a plane, while a ferritin film of one layer is formed of multiple ferritin 15 molecules as viewed in a cross section.

The array in which a ferritin film of two or more layers is formed as shown in the cross-sectional view in FIG. 2 is not included in the arrays referred to by the term “two-dimensional array”. Such an array is referred to as “three-dimensional array” if necessary, and is distinguished from the term “two-dimensional array” herein. However, exclusion of the two-dimensional arrays having the ferritin film of one layer with the three-dimensional array just in part (i.e., locally) from the term “two-dimensional array” is not intended.

The surface of the substrate is hydrophilic. As the substrate, a Si substrate can be used.

By oxidizing the surface of the Si substrate to give SiO₂, hydrophilicity can be imparted to the surface. In this case, the surface of the substrate will have a slightly negative potential.

By covering the surface of the substrate with 3-aminopropyltriethoxysilane (hereinafter, may be also referred to as “APTES”), the hydrophilicity can be imparted to the surface of the substrate. In this case, the surface of the substrate has a slightly positive potential.

The method of two-dimensionally arraying ferritin on a substrate according to the present invention has a development step and a removal step. The development step is explained first.

(1) Development Step

In the development step, a solution that contains a solvent, the ferritin as described above, and 2 mM to 100 mM ammonium acetate is developed on the substrate.

The solution is typically a buffer, and a Tris buffer may be illustrated as its example. In this case, the solvent almost corresponds to water accounting for a major portion of the buffer.

When the buffer contains a metal ion, the metal ion shall remain on the substrate as an impurity following the two-dimensionally arraying of ferritin. Thus, problems also described in “SUMMARY OF THE INVENTION” can be caused.

Therefore, it is desired that the buffer does not include a metal ion. Also in this respect, a Tris buffer is preferred.

In adjusting the pH of the buffer, the problems of the metal ion can be also caused. When the pH is elevated, sodium hydroxide, potassium hydroxide or the like is generally used. It is probable that sodium, potassium or the like included in this agent may finally remain in the form of a salt on the substrate as an impurity.

Therefore, in adjusting the pH of the buffer, it is preferable to adjust the pH not from low to high, but from high to low. When the pH is adjusted from high to low, hydrochloric acid may be used. Hydrochloric acid does not include any metal ion.

When adjustment of the pH from low to high is required despite the intention, it is desired that the amount of sodium hydroxide, potassium hydroxide to be used is minimized.

The solution contains 2 mM to 100 mM ammonium acetate ((CH₃COONH₄).

When the concentration of ammonium acetate is less than 2 mM, ferritin is irregularly dispersed on the substrate as demonstrated in Comparative Examples described later and corresponding photographs. Therefore, regular two-dimensional array of ferritin is not attained.

When the concentration of ammonium acetate exceeds 100 mM, ferritin is irregularly aggregated on the substrate as demonstrated in Comparative Examples described later and corresponding photographs. Therefore, regular two-dimensional array of ferritin is not attained.

Specific examples of the process for the development include the following processes in addition to the process of dropwise addition of the solution on the substrate. More specifically, the solution is added dropwise on a substrate of a thin film typified by Parafilm, and then the substrate is calmly placed on the solution with the hydrophilic face down. Accordingly, the solution is sandwiched between the thin film typified by Parafilm and the substrate with the hydrophilic face down.

(2) Removal Step

Next, the removal step is explained. In the removal step, the solvent is removed from the solution which had been developed on the substrate. Because the solution is typically a buffer, the solvent will be almost water accounting for a major portion of the buffer. Hence, the process for removing water from the substrate is explained in this section.

Specific examples of the process for removing the solvent include a process in which the substrate is subjected to centrifugal separation, as well as a process in which the solvent is evaporated from the substrate. In light of rapid removal of the solvent, the process in which the substrate is subjected to centrifugal separation is preferred. In any case, the process is acceptable as long as water is removed from the substrate in the removal step, which may include drying and concentration, irrespective of the procedure.

In the manner described above, ferritin can be two-dimensionally arrayed on the substrate. When the quantum dot is to be obtained, in general, thus two-dimensionally arrayed ferritin is removed by heat and then metal oxide is reduced as needed, whereby the quantum dot of the metal two-dimensionally arrayed on the substrate can be readily obtained.

In this method, any metal ion for achieving linking between two adjacent ferritin is unnecessary, therefore, an adverse effect which may be caused by the metal ion (for example, generation of an unexpected interface state and the like) can be suppressed.

Also, the metal can be substituted with a compound semiconductor (see, pamphlet of International Publication No. 03/099008).

### EXAMPLES

Hereinafter, the present invention will be explained in more detail by way of Examples. In the present Experimental Examples, the reagents listed in the Table 1 below were used.

TABLE 1

| Abbrevia- | | | | |
|---|---|---|---|---|
| tion | Trade name | Cat. No. | lot No. | |
| Tris | Trizma base | 76066-500G | 025K5432 | SIGMA- |
| | | | | ALDRICH |
| HEPES | HEPES | 342-01375 | SF076 | DOJINDO |
| | | | | Laboratories |
| AIS | Ammonium | 091-00855 | CEK7339 | Wako Pure |
| | iron (II) | | | Chemical |
| | sulfate | | | Industries, |
| | hexahydrate | | | Ltd. |
| Indium | Indium (III) | 20020-32 | 408C2100 | Wako Pure |
| sulfate | sulfate | | | Chemical |
| | | | | Industries, |
| | | | | Ltd. |
| NaH₂PO₄ | Sodium | 197-09705 | CEJ1855 | Wako Pure |
| | dihydrogen- | | | Chemical |
| | phosphate | | | Industries, |
| | (anhydride) | | | Ltd. |
| NH₃ | 1 N aqueous | 01793-08 | | KANTO |
| | ammonia | | | CHEMICAL |
| | | | | CO., INC. |
| Ammonium | Ammonium | 019-02835 | AST2519 | Wako Pure |
| acetate | acetate | | | Chemical |
| | | | | Industries, |
| | | | | Ltd. |
| APTES | 3-aminopropyl- | KBE-903 | | Shin-Etsu |
| | triethoxy- | | | Silicones |
| | silane | | | |

### (Preparation 1: CNHB-Fer0 Abundant Expression, Purification)

First, synthesis and purification procedures of apoCNHB-Fer0 are demonstrated below.

1. A plasmid vector pKIS2 (SEQ ID NO: 3) for protein expression was introduced into Escherichia coli XL1-blue (NOVAGENE), to execute transformation (see, also ECOS TM Competent E. coli DH5α, JM109, XL1-Blue, BL21 (DE3) Manual (ver.6) provided by NIPPON GENE CO., LTD.).

2. A colony of the transformed Escherichia coli was subjected to shaking culture (apparatus: TAITEC Bio Shaker BR-40LF, present temperature: 37° C., culture period: 5 to 7 hrs, shaking speed: 120 rpm) in 1 ml of an LB medium containing 50 μg/ml ampicillin charged in a 15 ml sterile Corning tube.

3. The aforementioned culture solution (0.1 to 0.5 ml) was subjected to shaking culture in 50 ml of an LB medium containing 50 μg/ml ampicillin in a 500-ml Erlenmeyer flask at 37° C. for 16-20 hrs.

4. Turbidity of the medium was measured with a spectrophotometer (Ultrospec 3100 pro, GE Healthcare Biosciences). When OD600 reached to 0.1 to 0.5, 50 ml of the aforementioned culture solution was subjected to spinner culture (apparatus: ABLE BMS-10/05, present temperature: 37° C., stirring speed: shaking speed: 200 rpm, air flow rate: 4 L/min, culture period: 18 to 20 hrs) in 6 L of an LB medium containing 100 μg/ml ampicillin.

5. Turbidity of the medium was measured, and was confirmed as OD600: 4.0 to 5.0. The bacteria were harvested using a low speed centrifuge (model: Avanti HP-25, rotor number: JA-10, Beckman Inc,; preset temperature: 4° C., preset number of revolutions: 8000 rpm, time: 10 min) in a centrifuge tube for JA-10.

6. The harvested bacteria were suspended in 50 mM Tris-HCl (200 ml to 300 ml), and collected in a centrifuge tube for JA-10 using the low speed centrifuge (the same as that in the above section 5).

7. The harvested bacteria were suspended in 50 mM Tris-HCl (120 ml), stood in ice, and the cells were disrupted with an ultrasonicator (apparatus: Branson Digital Sonifier 450, preset output: 140 W, pulse preset :on/off one sec, disruption time: 2 min×3 times).

8. The mixture was centrifuged with a low speed centrifuge (model: Avanti HP-25, rotor number: JA-20, Beckman Inc,; preset temperature: 4° C., preset centrifugal force: 6000×g, time: 10 min), and the supernatant was collected.

9. The collected supernatant was subjected to a heat treatment (75° C., 20 min), and following the heat treatment, it was left to stand at room temperature until the temperature returned to the ordinary temperature (approximately 1 hour).

10. The treated liquid was centrifuged with a low speed centrifuge (the same as that in the above section 8), and the supernatant was collected.

11. To the collected supernatant was added 5 M NaCl to give the final concentration of 0.5 M NaCl, followed by being suspended therein.

12. The suspension was centrifuged with a low speed centrifuge (the same as that in the above section 8), and the precipitate was collected.

13. The collected precipitate was suspended in 50 mM Tris-HCl (120 ml), to which 10.54 ml of 5 M NaCl was added to give the final concentration of 0.4 M NaCl, followed by being suspended therein.

14. The suspension was centrifuged with a low speed centrifuge (the same as that in the above section 8), and the precipitate was collected.

15. The precipitate was collected, and the manipulations of 13 to 14 were repeated again.

16. The precipitate was suspended in 50 mM Tris-HCl (60 ml), and the suspension was passed through a 0.22 μm syringe filter, thereby completing the purification.

### (Preparation 2: Determination of CNHB-Fer0 Concentration)

The concentration of the protein solution (solution containing CNHB-Fer0) obtained in the aforementioned section: CNHB-Fer0 Abundant Expression, Purification, is unknown.

Thus, according to the following process, concentration of the protein solution having an unknown concentration was determined.

In the determination of the protein concentration, a DC protein assay kit (Cat. No. 500-0112JA, BioRad) was used according to a Lowry method.

1. As a standard protein, a BSA (Bovine Albumin Serum, Cat. No. 23209, PIACE) solution having a known concentration was used after diluting to predetermined concentrations (0.2, 0.4, 0.6, 1.0, 2.0 mg/ml) in ultrapure water.

2. The reaction mixture was produced in the following procedures. The protein solution (or ultrapure water as a control) in a volume of 25 μl and 125 μl of reagent A were placed in a microtube, and then mixed.

3. Subsequently, 1 ml of reagent B was placed on the same microtube and mixed, whereby the reaction was allowed at room temperature of 25 (±1)° C. for 15 min or longer.

4. After the reaction, the absorbance was measured within 1 hour with a spectrophotometer (Ultrospec 3100 pro, GE Healthcare Biosciences) at 750 nm.

5. The absorbance at 750 nm was plotted with respect to the protein concentration of the BSA solutions, and the formula: (protein concentration of unknown sample)=A (absorbance at 750 nm of unknown sample)+C was derived according to a least square method.

6. The arbitrarily diluted solution of the sample was subjected to determination of the protein concentration according to the aforementioned procedures, and the concentration of the sample stock solution was derived by multiplying by the dilution factor. Thus derived protein concentration (concentration of CNHB-Fer0 included in the solution) was 10.56 mg/ml.

(Preparation 3: Purity Test of apoCNHB-Fer0)

Purity of the resulting apoCNHB-Fer0 was tested as to whether it is suited for core synthesis, according to the following procedures.

The purity was determined by gel filtration as in the following.

1. HPLC (L-6210, Hitachi, Ltd.) was used to which a TSK-GEL BIOASSIST G4SWXL column (Tosoh Corporation) was connected.

2. Using 50 ml or more 50 mM Tris HCl buffer, pH 8.0 as a mobile phase, the liquid had been fed beforehand at a flow rate of 1.0 ml per min.

3. The purified solution having a concentration of 1 mg/ml in a volume of 0.1 ml was loaded to a sample loop, and injected into the column at a flow rate of 1.0 ml per min.

4. Monitoring was carried out with a UV/VIS detector (L-4200, Hitachi, Ltd.) at a wavelength of 280 nm, and recorded on a Chromato-integrator (D-2600, Hitachi, Ltd.).

5. It was ascertained that only peaks derived from apoCNHB-Fer0 (monomer: 8.6 min, dimer: 7.8 min) were found, and the peaks which corresponded to the CNHB-Fer0 subunits included in the sample (elution time: 13 to 14 min) were below the detection limit.

### (Preparation 4-1: Synthesis of CNHB-Fer0 Including in Therein)

In oxide for use in production of two-dimensional array was synthesized inside apoCNHB-Fer0 as described below.

In this Example, 80 ml of a reaction mixture was prepared according to the following procedures such that the final solution composition includes 0.2 M sodium dihydrogenphosphate, 12 mM ammonia, 40 mM HCl, 0.1 mg/ml apoCNHB-Fer0, and 1 mM in diumsulfate.

1. To a 300 ml disposable beaker were added 16 ml of 1 M sodium dihydrogenphosphate, 0.96 ml of 1 M ammonia, 3.2 ml of 1 N HCl, and 59.082 ml of ultrapure water in this order, and the mixture was stirred with a stirrer bar.

2. The pH was measured with a pH meter, and the pH of 2.88 (within ±0.02) was determined.

3. Thereto was added a 2 mM Tris (pH 8.0) solution containing 0.758 ml of 10.56 mg/ml apoCNHB-Fer0, and the mixture was stirred with a stirrer bar.

4. Thereto was added 41.4 mg of indium sulfate powder to dissolve the powder in the reaction mixture.

5. The beaker charged with the reaction mixture was covered by a Saran Wrap (trade mark, a thin plastic wrap), and the reaction was allowed at 25° C. (±1° C.) for 3 hrs while stirring.

6. After the reaction, each 40 ml of the reaction mixture was dispensed into a 50 ml Falcon tube.

7. The Falcon tubes were placed in a swing rotor of a centrifuge LC-200 (TOMY), and centrifuged at 3000 rpm for 10 min. Supernatant 1 was removed, and precipitate 1 was collected.

8. To the precipitate 1 was added 5 ml of 50 mM Tris HCl buffer (pH 8.0), followed by being suspended using a vortex mixer.

9. The Falcon tube including the precipitate 1 was placed in a swing rotor of a centrifuge LC-200, and centrifuged at 3000 rpm for 10 min to obtain a supernatant 2 and a precipitate 2. The supernatant 2 was dispensed into a new Falcon tube.

10. To the precipitate 2 was added 5 ml of 50 mM Tris HCl buffer (pH 8.0), followed by being suspended using a vortex mixer.

11. The Falcon tube including the precipitate 2 was placed in a swing rotor of a centrifuge LC-200, and centrifuged at 3000 rpm for 10 min to obtain supernatant 2′ and precipitate 2′. The supernatant 2′ was dispensed into a new Falcon tube.

12. To the precipitate 2′ was added 5 ml of 50 mM Tris HCl buffer (pH 8.0), followed by being suspended using a vortex mixer.

13. The Falcon tube including the precipitate 2′ was placed in a swing rotor of a centrifuge LC-200, and centrifuged at 3000 rpm for 10 min to obtain a supernatant 2″ and a precipitate 2″. The supernatant 2″ was dispensed into a new Falcon tube.

14. To each of the supernatant 2 (about 5 ml), the supernatant 2′ (about 5 ml), and the supernatant 2″ (about 5 ml) was added 0.5 ml of 5 M NaCl. The Falcon tubes were then inverted, whereby the mixture was stirred. The tubes were left to stand at 4° C. (±1° C.) for 3 hrs.

15. The Falcon tubes were placed in a swing rotor of a centrifuge LC-200, and centrifuged at 3000 rpm for 10 min. Supernatant 3, supernatant 3′ and supernatant 3″ were removed, and precipitate 3, precipitate 3′ and precipitate 3″ were collected.

16. To the precipitate 3 was added 10 ml of 50 mM Tris HCl buffer (pH 8.0), followed by being suspended using a vortex mixer to obtain suspension 3.

17. To the precipitate 3′ was added the suspension 3, followed by being suspended using a vortex mixer to obtain suspension 3′.

18. To the precipitate 3″ was added 10 ml of the suspension 3′, followed by being suspended using a vortex mixer to obtain suspension 3″.

19. To the suspension 3″ (about 10 ml) was added 0.9 ml of 5 M NaCl, and the Falcon tube was inverted, whereby the mixture was stirred.

20. The Falcon tube was placed in a swing rotor of a centrifuge LC-200, and centrifuged at 3000 rpm for 10 min. Supernatant 4 was removed, and precipitate 4 was collected.

21. To the precipitate 4 was added 10 ml of 50 mM Tris HCl buffer (pH 8.0), followed by being suspended using a vortex mixer to obtain suspension 4.

22. Suspension 5 was transferred to a collection tube of an Apollo 20 ml (QMWL 150 kDa) centrifugal concentrator.

23. The Apollo 20 ml centrifugal concentrator was placed in a swing rotor of a centrifuge LC-200, and the solution was concentrated by repeating the centrifugation at 3000 rpm until the volume of the solution left in the collection tube became 1 ml or less.

24. Concentrated solution 1 was taken from the collection tube.

25. The concentration of CNHB-Fer0 having In oxide as a core (hereinafter, denoted as CNHB-Fer0(In)) was determined according to the procedures demonstrated in the “Preparation 2: Determination of CNHB-Fer0 Concentration”.

### (Preparation 4-2: Synthesis of CNHB-Fer0 Including Fe Therein)

Fe oxide for use in production of two-dimensional array was synthesized inside apoCNHB-Fer0 as described below.

In this Example, 80 ml of a reaction mixture was prepared according to the following procedures such that a final solution composition includes 80 mM HEPES pH 7.5, 0.5 mg/ml apoCNHB-Fer0, and 5 mM (NH₄)₂Fe(SO₄)₂.

1. To 125 ml square medium bottle (Nalge Nunc International K.K.: 2019-0125) were added the following solutions in the indicated order. The bottle was rotated in a horizontal direction to allow the solution to be stirred.

12.8 ml of 0.5 M HEPES pH 7.5; 55.4 ml of ultrapure water; 3.8 ml of a 2 mM Tris (pH 8.0) solution containing 10.56 mg/ml apoCNHB-Fer0.

2. To 20 ml of ultrapure water which had been chilled to 8° C. for 1 hour or longer was added 0.392 g of ammonium sulfate iron powder to prepare a 50 mM ammonium sulfate iron solution.

3. To the square medium bottle including the reaction mixture described above was added 8 ml of 50 mM ammonium sulfate iron solution. The bottle was rotated in a horizontal direction to allow the solution to be stirred. The reaction was allowed in a 8° C. (±1° C.) refrigerator for 18 hrs.

4. After the reaction, each 40 ml of the reaction mixture was dispensed into two 50 ml Falcon tubes.

5. Each Falcon tube was placed in a swing rotor of a centrifuge LC-200, and centrifuged at 3000 rpm for 10 min. Supernatant 1 was collected in a new Falcon tube.

6. To the supernatant 1 (about 40 ml×2) was added each 4 ml of 5 M NaCl, and the two Falcon tubes were inverted, whereby the mixtures were stirred.

7. Each Falcon tube was placed in an angle rotor of a centrifuge MX-300 (Kubota), and centrifuged at 10000 rpm for 10 min. Supernatant 2 was removed, and precipitate 2 was collected.

8. To each precipitate 2 was added 3 ml of 50 mM Tris HCl buffer (pH 8.0), followed by being suspended using a vortex mixer to obtain suspension 2 (about 3 ml×2).

9. Each Falcon tube was placed in an angle rotor of a centrifuge MX-300, and centrifuged at 10000 rpm for 10 min. Precipitate 3 was removed, and at the same time, supernatant 3 (about 6 ml) was collected in a new Falcon tube.

10. To the supernatant 3 (about 6 ml) was added 0.6 ml of 5 M NaCl, and the Falcon tube was inverted, whereby the mixture was stirred.

11. The Falcon tube was placed in an angle rotor of a centrifuge MX-300, and centrifuged at 10000 rpm for 10 min. Supernatant 4 was removed, and precipitate 4 was collected.

12. To the precipitate 4 was added 5 ml of 50 mM Tris HCl buffer (pH 8.0), followed by being suspended using a vortex mixer to obtain suspension 4.

13. The Falcon tube including the suspension 4 was placed in an angle rotor of a centrifuge MX-300, and centrifuged at 10000 rpm for 10 min. Precipitate 5 was removed, and supernatant 5 was collected in a new Falcon tube.

14. To the supernatant 5 (about 5 ml) was added 0.5 ml of 5 M NaCl, and the Falcon tube was inverted, whereby the mixture was stirred.

15. The Falcon tube including the suspension 5 was placed in an angle rotor of a centrifuge MX-300, and centrifuged at 3000 rpm for 10 min. Supernatant 6 was removed, and precipitate 6 was collected.

16. To the precipitate 6 was added 3 ml of 50 mM Tris HCl buffer (pH 8.0), followed by being suspended using a pipette to obtain suspension 6.

17. The suspension 6 was transferred to a collection tube of an Apollo 20 ml (QMWL 150 kDa) centrifugal concentrator.

18. The Apollo 20 ml centrifugal concentrator was placed in a swing rotor of a centrifuge LC-200, and the solution was concentrated by repeating the centrifugation at 3000 rpm until the volume of the solution left in the collection tube became 1 ml or less to obtain concentrated solution 1.

19. Concentrated solution 1 was taken from the collection tube.

20. The concentration of CNHB-Fer0 having Fe oxide as a core (hereinafter, denoted as CNHB-Fer0(Fe)) was determined according to the procedures demonstrated in the “Preparation 2: Determination of CNHB-Fer0 Concentration”.

### (Preparation 5: High Purification of CNHB-Fer0(X))

A highly purified (monomer purity: 99.5% or greater) CNHB-Fer0 having core inside (hereinafter, denoted as CNHB-Fer0(X), wherein is In or Fe) is desired for two-dimensional arraying.

Thus, in this Example, CNHB-Fer0(X) for use in two-dimensional arraying was highly purified as demonstrated below.

1. A Tricorn 10/600 column (GE Healthcare) packed with a TSK-GEL BIOASSIST G4SWXL resin (Tosoh Corporation) was connected to HPLC (L-6210, Hitachi, Ltd.).

2. Using 100 ml or more 50 mM Tris HCl buffer, pH 8.0 as a mobile phase, the liquid had been fed beforehand at a flow rate of 0.5 ml per min.

3. The concentrated solution 1 in a volume of 3 ml or less was loaded to a sample loop, and injected into the column at a flow rate of 0.5 ml per min.

4. Monitoring was carried out with a UV/VIS detector (L-4200, Hitachi, Ltd.) at a wavelength of 280 nm, and recorded on a Chromato-integrator (D-2600, Hitachi, Ltd.).

5. Each 0.5 ml of the eluate was collected with a fraction collector (Waters Corporation), and the fraction containing a CNHB-Fer0(X) monomer was collected.

6. The concentration of CNHB-Fer0(X) was determined according to the procedures demonstrated in the “Preparation 2: Determination of CNHB-Fer0 Concentration”.

(Preparation 6: Removal of apo CNHB-Fer0 from CNHB-Fer0(X) Solution)

The two-dimensional arraying requires CNHB-Fer0(X) having a core formation rate of 90% or higher. When the core formation rate is not higher than 90%, a step of elevating the core formation rate is carried out according to the following procedures. Thus, apo CNHB-Fer0 was removed from the CNHB-Fer0(X) solution for use in the two-dimensional arraying, through density gradient centrifugation as described below.

1. Glycerol and 1 M Tris HCl, pH 8.0 were mixed to give the composition shown in Table 2 below to prepare 60, 30, 15% (w/v) glycerol solutions.

TABLE 2

| | Glycerol (g) | 1 M TrisHCl pH 8.0 (ml) | Ultrapure water (ml) |
|---|---|---|---|
| 60% | 60 | 2 | 38 |
| 30% | 30 | 2 | 68 |
| 15% | 15 | 2 | 83 |

2. Centrifuge tubes (Parts No. 326823, BECKMAN COOULTER) were placed horizontally, and therein 10 ml, 10 ml and 15 ml of 60%, 30%, 15% (w/v) glycerol solutions, respectively were overlaidgently from the bottom of the tube.

3. The sample in a volume up to about 3 ml was overlaid on the glycerol solution, and was inserted into a packet of a SW-28 swing rotor (BECKMAN COOULTER). Weight of the packets at the opposing corner was balanced, respectively, and the packets were calmly hanged in the rotor body.

4. The SW-28 swing rotor was placed in an Optima L-80P centrifuge (BECKMAN COOULTER), and centrifuged at 4° C. and 20,000 rpm for 20 hrs.

5. After completing the centrifugation, the centrifuge tubes were removed from the centrifuge. The bottom of the tube was punctured with a needle (Terumo 20 G or 18 G), and the solution was quickly received into a macrotest tube.

6. The solution was dispensed into about 1 ml each, whereby 20 fractions were collected. The absorbance (540 nm for Fecore, and 280 nm for In core) of each fraction was measured with a spectrophotometer (Ultrospec 3100 pro, GE Healthcare Biosciences), and the fractions were collected until maximum absorbance was found.

7. To a collection tube of Apollo 20 ml (QMWL 150 kDa) centrifugal concentrator was transferred the aforementioned fractions.

8. The Apollo 20 ml centrifugal concentrator was placed in a swing rotor of a centrifuge LC-200.

9. The solution was concentrated until the glycerol concentration became 1/1000 or lower by repeating dilution with 2 mM Tris buffer and centrifugation at 3000 rpm, whereby concentration was achieved until the volume of the solution left in the collection tube became 1 ml or less.

10. The concentration of CNHB-Fer0(X) was determined according to the procedures demonstrated in the “Preparation 2: Determination of CNHB-Fer0 Concentration”.

### (Preparation 7: Preparation of Hydrophilized Substrate)

The two-dimensional arraying requires a substrate having a hydrophilic surface.

Hereinafter, procedures for producing a thermally-oxidized silicon substrate, a hydrophilizing vapor-deposited carbon substrate and an APTES-modified substrate are demonstrated. Any of these substrates has a hydrophilic surface.

(Thermally-Oxidized Silicon Substrate)

Procedures for hydrophilizing a substrate surface through UV/O₃ washing (washing with ultraviolet ray/ozone) to remove organic matters on the surface are demonstrated below.

1. Just before (i.e., immediately before allowing for two-dimensionally arraying of ferritin as described later), a thermally-oxidized silicon substrate (SiO₂ film thickness: 3 nm) was cleaved into a piece of 5×10 mm.

2. Using an apparatus (Model UV-1, SAMCO, Inc.), UV/03 washing of the thermally-oxidized silicon substrate was carried out at a substrate temperature of 110° C., and an oxygen flow rate of 0.5 L/min for a washing time of 10 min.

(Hydrophilizing Vapor-Deposited Carbon Substrate)

Procedures for hydrophilizing a substrate surface through vacuum deposition of carbon on the thermally-oxidized silicon substrate followed by an atmospheric plasma treatment are demonstrated below.

1. The thermally-oxidized silicon substrate (SiO₂ film thickness: 3 nm) was cleaved into a piece of 5×10 mm.

2. Using an apparatus (Model UV-1, SAMCO, Inc.), UV/O₃ washing of the thermally-oxidized silicon substrate was carried out at a substrate temperature of 110° C., and an oxygen flow rate of 0.5 L/min for a washing time of 10 min.

3. Carbon was vacuum-deposited (JEE-420, JEOL Ltd.) to give a thickness of 10 nm or greater on the thermally-oxidized silicon substrate.

4. Just before (i.e., immediately before allowing for two-dimensionally arraying of ferritin as described later), an ambient air plasma treatment was carried out with a hydrophilizing treatment apparatus (HDT400 JEOL Ltd.).

(APTES-Modified Substrate)

Procedures for modifying the substrate surface with APTES through exposing the thermally-oxidized silicon substrate to vapor are demonstrated below.

1. The thermally-oxidized silicon substrate (SiO₂ film thickness: 3 nm) was cleaved into a piece of 5×10 mm, and washed with running water (5 min).

2. Using an apparatus (Model UV-1, SAMCO, Inc.), UV/O₃ washing of the thermally-oxidized silicon substrate was carried out at a substrate temperature of 110° C., and an oxygen flow rate of 0.5 L/min for a washing time of 10 min.

3. Since APTES (liquid) has been refrigerated, the reagent bottle was removed prior to carrying out the experiment, and allowed to stand to warm up to the room temperature over one hour.

4. A glass dish, an aluminum plate, an aluminum cup, and a jig used in the experiment were subjected to nitrogen blowing just before use.

5. The aluminum cup for charging APTES, and the jig for placing the thermally-oxidized silicon substrate were set on the aluminum plate placed on a clean glass dish.

6. The washed thermally-oxidized silicon substrate was placed on the jig.

7. APTES in an amount of 0.5 ml was charged in the aluminum cup with a dropping pipette.

8. The glass dish was closed with a lid, and doubly sealed with Parafilm.

9. The thermally-oxidized silicon substrate was exposed to the APTES vapor for 3 hrs or longer and 24 hrs and shorter at a room temperature.

10. After the reaction, the dish was opened, and the substrate was washed according to the following procedures.

11. To three 500-ml beakers which had washed with the same solvent for use, i.e., dehydrated ethanol was poured 100 ml of dehydrated ethanol.

12. The APTES-modified substrate including the jig all together was immersed in dehydrated ethanol, and gently shaken to wash the substrate surface.

13. The solution was quickly changed to fresh dehydrated ethanol so as to prevent the surface from drying. This operation was repeated three times in a similar manner.

14. Finally, it was washed with running water (5 min), and the substrate was dried with a spin coater.

(Two-Dimensional Array of Ferritin)

After completing the foregoing Preparations 1 to 7, ferritin was two-dimensionally arrayed according to the procedures below (hereinafter, may be referred to as “sandwich method”).

1. The protein having a final concentration being 2× concentrated, and 2 mM Tris buffer were provided. For example, when the final concentration is 0.5 mg/ml CNHB-Fer0(Fe), 1.0 mg/ml CNHB-Fer0(Fe) was provided.

2. A solution for arraying having a final concentration being 2× concentrated was provided. For example, in the case of ammonium acetate having a final concentration of 20 mM, a 40 mM ammonium acetate solution was provided.

3. Each 5 μl of the protein solution, and the arraying solution was charged in a micro test tube, and mixed by pipetting or Vortex mixture.

4. Parafilm having an arbitrary size was placed in a plastic dish, and 5 μl of the mixed solution was dropped on the Parafilm.

5. The hydrophilizing treatment surface of the substrate prepared according to any one of “(Preparation 7: Preparation of Hydrophilized Substrate)” was placed so as to contact with the droplet.

6. The plastic dish was covered by a lid, and left to stand in an incubator (LTI-2000, TOKYO RIKAKAICO, LTD) at 20 (±0.5)° C. for 30 min.

7. After a predetermined time period, the substrate was peeled off from the Parafilm with vacuum tweezers, and transferred to a 1.5 ml micro test tube.

8. The aforementioned micro test tube was centrifuged (5415D eppendrf) at 1500G for 10 min, whereby excess solution on the substrate was removed.

9. The substrate was removed from the micro test tube, and observed with SEM (JEOL SEM7400F). The observation conditions were accelerating voltage of 5 kV, and emission electric current of 10 μA.

The results are as described below.

### Example 1

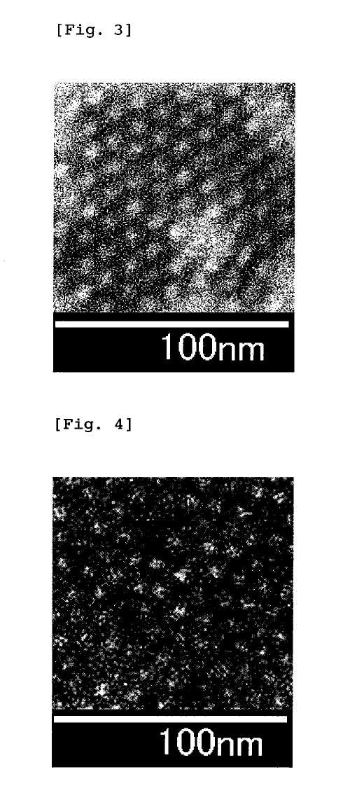

FIG. 3 shows a photograph illustrating the appearance of a two-dimensional array of ferritin obtained using 0.5 mg/ml CNHB-Fer0(In), 2 mM ammonium acetate, and the thermally-oxidized silicon substrate.

### Example 2

FIG. 4 shows a photograph illustrating the appearance of a two-dimensional array of ferritin obtained using 0.5 mg/ml CNHB-Fer0(In), 10 mM ammonium acetate, and the thermally-oxidized silicon substrate.

### Example 3

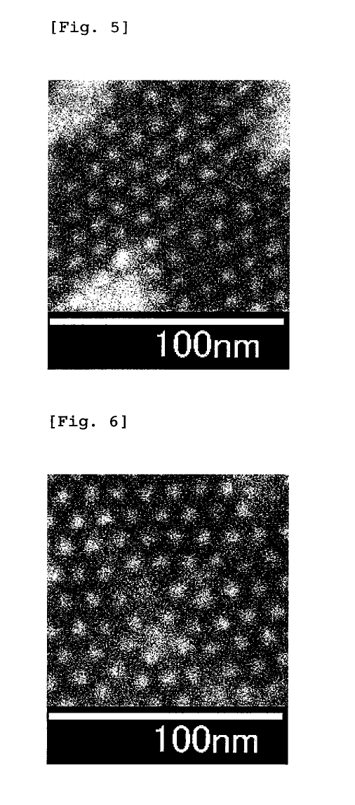

FIG. 5 shows a photograph illustrating the appearance of a two-dimensional array of ferritin obtained using 0.5 mg/ml CNHB-Fer0(In), 20 mM ammonium acetate, and the thermally-oxidized silicon substrate.

### Example 4

FIG. 6 shows a photograph illustrating the appearance of a two-dimensional array of ferritin obtained using 0.25 mg/ml CNHB-Fer0(In), 50 mM ammonium acetate, and the thermally-oxidized silicon substrate.

### Example 5

FIG. 7 shows a photograph illustrating the appearance of a two-dimensional array of ferritin obtained using 0.5 mg/ml CNHB-Fer0(In), 100 mM ammonium acetate, and the thermally-oxidized silicon substrate.

### Example 6

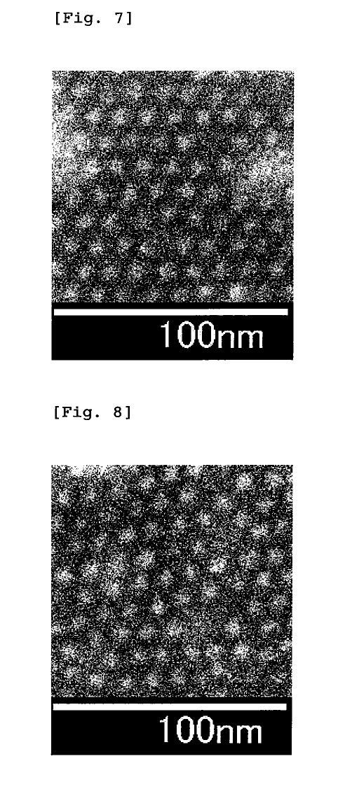

FIG. 8 shows a photograph illustrating the appearance of a two-dimensional array of ferritin obtained using 0.25 mg/ml CNHB-Fer0(In), 20 mM ammonium acetate, and the thermally-oxidized silicon substrate.

### Example 7

FIG. 9 shows a photograph illustrating the appearance of a two-dimensional array of ferritin obtained using 1.0 mg/ml CNHB-Fer0(Fe), 20 mM ammonium acetate, and the thermally-oxidized silicon substrate.

### Example 8

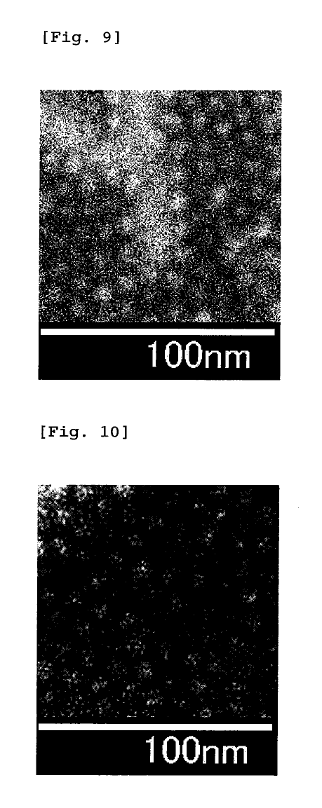

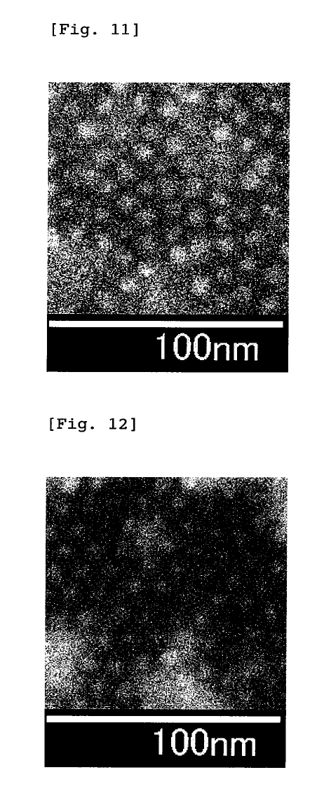

FIG. 10 shows a photograph illustrating the appearance of a two-dimensional array of ferritin obtained using 0.5 mg/ml CNHB-Fer0(Fe), 2 mM ammonium acetate, and the thermally-oxidized silicon substrate.

### Example 9

FIG. 11 shows a photograph illustrating the appearance of a two-dimensional array of ferritin obtained using 0.5 mg/ml CNHB-Fer0(Fe), 10 mM ammonium acetate, and the thermally-oxidized silicon substrate.

### Example 10

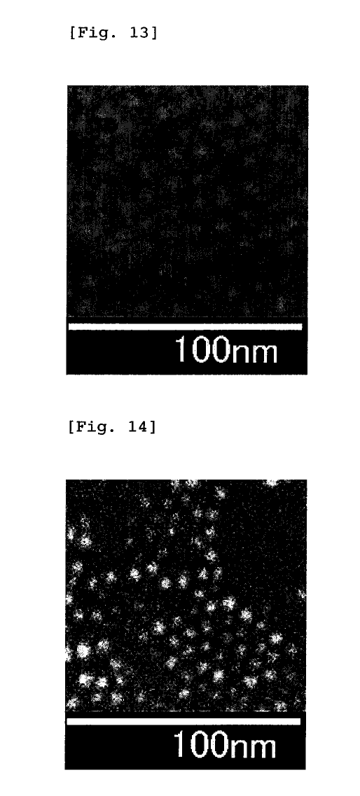

FIG. 12 shows a photograph illustrating the appearance of a two-dimensional array of ferritin obtained using 0.5 mg/ml CNHB-Fer0(In), 20 mM ammonium acetate, and the thermally-oxidized silicon substrate.

### Example 11

FIG. 13 shows a photograph illustrating the appearance of a two-dimensional array of ferritin obtained using 0.5 mg/ml CNHB-Fer0(In), 13 mM ammonium acetate, and the thermally-oxidized silicon substrate.

### Example 12

FIG. 14 shows a photograph illustrating the appearance of a two-dimensional array of ferritin obtained using 0.5 mg/ml CNHB-Fer0(Fe), 10 mM ammonium acetate, and the APTES-modified substrate.

As shown in from FIG. 3 to FIG. 14, ferritin can be two-dimensionally arrayed in a regular manner by using the factors shown in (a) to (c): (a) ferritin having the amino acid sequence set out in SEQ ID NO: 1 on the outer peripheral surface; (b) a substrate having a hydrophilic surface; and (c) ammonium acetate having a concentration of 2 mM to 100 mM.

### Comparative Example 1

FIG. 15 shows a photograph illustrating the appearance of ferritin on the substrate obtained using 0.5 mg/ml CNHB-Fer0(In), 500 mM ammonium acetate, and the thermally-oxidized silicon substrate.

In FIG. 15, two-dimensional array of ferritin could not be verified because the concentration of ammonium acetate of 500 mM was too high.

### Comparative Example 2

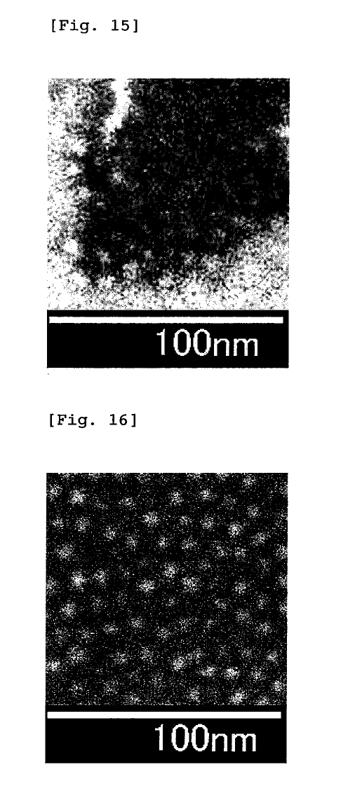

FIG. 16 shows a photograph illustrating the appearance of ferritin on the substrate obtained using 0.5 mg/ml CNHB-Fer0(In), pure water, and the thermally-oxidized silicon substrate.

In FIG. 16, regular arraying of ferritin could not be verified because ammonium acetate was not used, i.e., the solution did not include ammonium acetate.

### Comparative Example 3

FIG. 17 shows a photograph illustrating the appearance of ferritin on the substrate obtained using 0.5 mg/ml CNHB-Fer0(In), 1 mM Tris, and the thermally-oxidized silicon substrate.

In FIG. 17, regular arraying of ferritin could not be verified because ammonium acetate was not used, i.e., the solution did not include ammonium acetate.

### Comparative Example 4

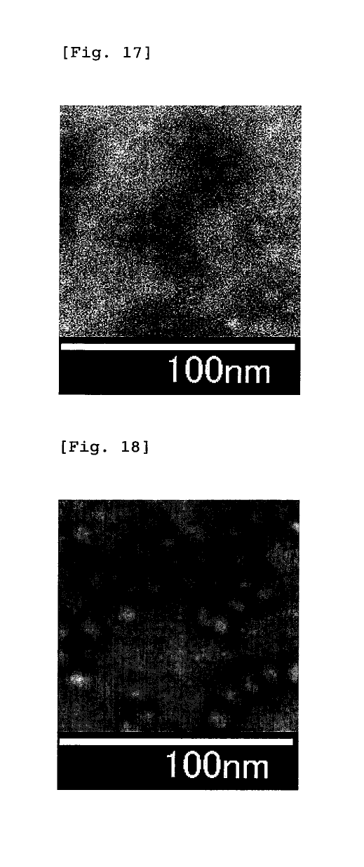

FIG. 18 shows a photograph illustrating the appearance of ferritin on the substrate obtained using 0.5 mg/ml CNHB-Fer0(Fe), 1 mM Tris, and the thermally-oxidized silicon substrate.

In FIG. 18, regular arraying of ferritin could not be verified because ammonium acetate was not used, i.e., the solution did not include ammonium acetate.

### Comparative Example 5

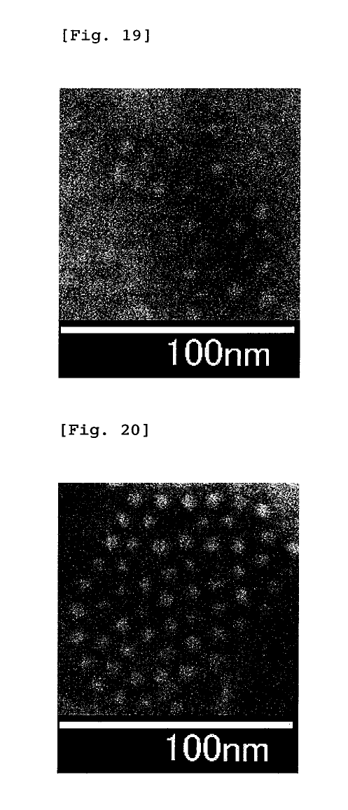

FIG. 19 shows a photograph illustrating the appearance of ferritin on the substrate obtained using 0.5 mg/ml Fer0(In), 20 mM ammonium acetate, and the thermally-oxidized silicon substrate.

In FIG. 19, regular arraying of ferritin could not be verified because simple ferritin was used not having the amino acid sequence set out in SEQ ID NO: 1 on the outer peripheral surface of ferritin.

### Comparative Example 6

FIG. 20 shows a photograph illustrating the appearance of ferritin on the substrate obtained using 0.5 mg/ml Fer0(In), 12.5 mM PIPES, and the thermally-oxidized silicon substrate.

In FIG. 20, regular arraying of ferritin could not be verified because simple ferritin was used not having the amino acid sequence set out in SEQ ID NO: 1 on the outer peripheral surface of ferritin, and the solution did not include ammonium acetate.

### Comparative Example 7

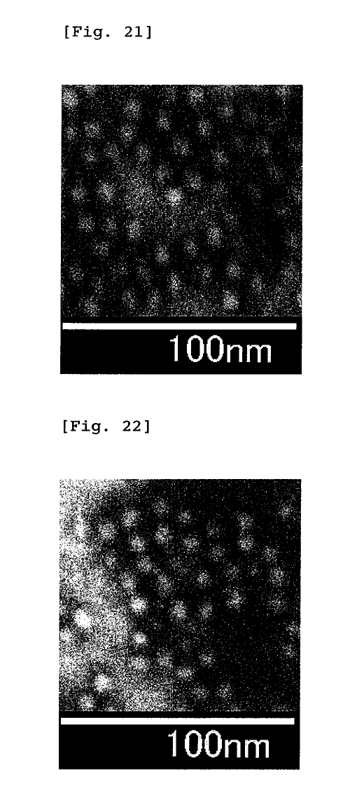

FIG. 21 shows a photograph illustrating the appearance of ferritin on the substrate obtained using 0.5 mg/ml Fer0(Fe), 12.5 mM PIPES, and the thermally-oxidized silicon substrate.

In FIG. 21, regular arraying of ferritin could not be verified because simple ferritin was used not having the amino acid sequence set out in SEQ ID NO: 1 on the outer peripheral surface of ferritin, and the solution did not include ammonium acetate.

### Comparative Example 8

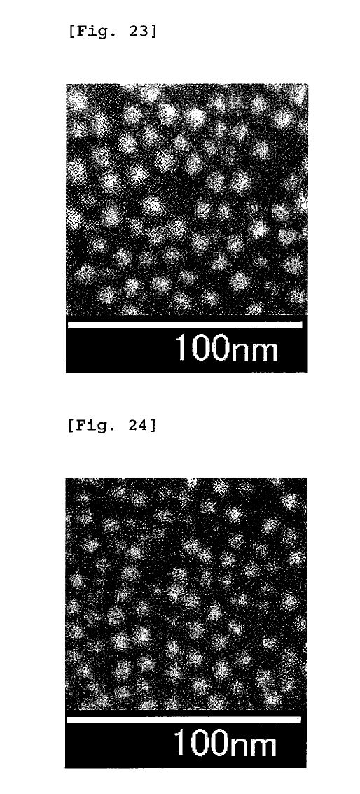

FIG. 22 shows a photograph illustrating the appearance of ferritin on the substrate obtained using 0.5 mg/ml Fer0(Fe), 50 mM PIPES, and the thermally-oxidized silicon substrate.

In FIG. 22, regular arraying of ferritin could not be verified because simple ferritin was used not having the amino acid sequence set out in SEQ ID NO: 1 on the outer peripheral surface of ferritin, and the solution did not include ammonium acetate.

### Comparative Example 9

FIG. 23 shows a photograph illustrating the appearance of ferritin on the substrate obtained using 0.5 mg/ml Fer0(Fe), 12.5 mM PIPES, and the hydrophilized carbon silicon substrate.

In FIG. 23, regular arraying of ferritin could not be verified because simple ferritin was used not having the amino acid sequence set out in SEQ ID NO: 1 on the outer peripheral surface of ferritin, and the solution did not include ammonium acetate.

### Comparative Example 10

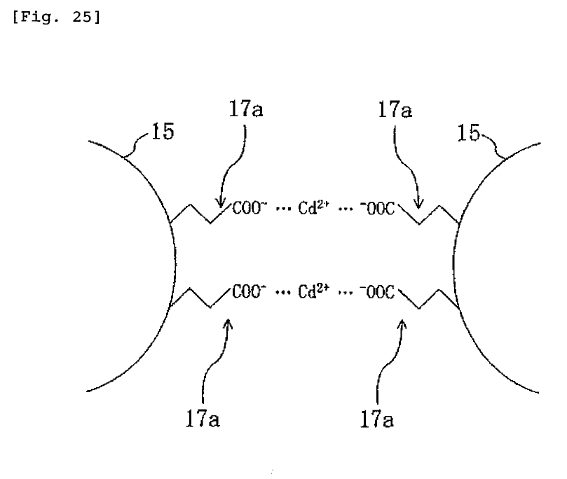

FIG. 24 shows a photograph illustrating the appearance of ferritin on the substrate obtained using 0.5 mg/ml Fer0(Fe), 50 mM PIPES, and the hydrophilized carbon silicon substrate.

In FIG. 24, regular arraying of ferritin could not be verified because simple ferritin was used not having the amino acid sequence set out in SEQ ID NO: 1 on the outer peripheral surface of ferritin, and the solution did not include ammonium acetate.

Also realized from FIG. 3 to FIG. 14, and from FIG. 15 to FIG. 24, for two-dimensional array of ferritin in a regular manner, it is essential to use (a) ferritin having the amino acid sequence set out in SEQ ID NO: 1 on the outer peripheral surface; (b) a substrate having a hydrophilic surface; and (c) ammonium acetate having a concentration of 2 mM to 100 mM.

According to the present invention, there is no metal ion for achieving linking between two adjacent ferritin. Therefore, any adverse effect typified by generation of an unexpected interface state in quantum dots composed of a two-dimensional array of a metal on a substrate can be suppressed.

The method of two-dimensionally arraying ferritin on a substrate according to the present invention does not require a metal ion for achieving linking between two adjacent ferritin, therefore, it can be applied to quantum dots expected for suppressing the adverse effect caused by the metal ion, and to semiconductor devices having such quantum dots.

What is claimed is:

1. A method of two-dimensionally arraying ferritin on a substrate, wherein
the ferritin has an amino acid sequence set out in SEQ ID NO: 1 on the outer peripheral surface;
the surface of the substrate is hydrophilic; and
the method comprises:
developing a solution that contains a solvent, the ferritin, and 2 mM to 100 mM ammonium acetate on the substrate; and
removing the solvent from the solution developed on the substrate.


SEQUENCE LISTING (3 sequences)

SEQ ID NO: 1 - protein, 12 aa, Artificial Sequence
  Twelve amino acids for alignment of ferritin on plate
DYFSSPYYEQLF

SEQ ID NO: 2 - protein, 187 aa, Artificial Sequence
  A modified horse ferritin having amino- terminal methionine and twelve amino acids at amino-terminal
MDYFSSPYYEQLFSSQIRQNYSTEVEAAVNRLVNLYLRASYTYLSLGFYFDRDDVALEFVCHFFRELAEEKREGAERLLKMQNQRGGRALFQDLQKPSQDEWGTTPDAMKAAIVLEKSLNQALLDLHALGSAQADPHLCDFLESHFLDEEVKLIKKMGDHLTNIQRLVGSQAGLGEYLFERLTLKHD

SEQ ID NO: 3 - DNA, 3846 bp, Artificial Sequence
  Plasmid vector for protein expression.
ctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagaattcaaattctatttcaaggagacaggatccatggattatttctcgagcccgtattatgaacagctgtttagctcccagattcgtcagaattattctactgaagtggaggccgccgtcaaccgcctggtcaacctgtacctgcgggcctcctacacctacctctctctgggcttctatttcgaccgcgacgatgtggctctggagggcgtatgccacttcttccgcgagttggcggaggagaagcgcgagggtgccgagcgtctcttgaagatgcaaaaccagcgcggcggccgcgccctcttccaggacttgcagaagccgtcccaggatgaatggggtacaaccccggatgccatgaaagccgccattgtcctggagaagagcctgaaccaggcccttttggatctgcatgccctgggttctgcccaggcagacccccatctctgtgacttcttggagagccacttcctagacgaggaggtgaaactcatcaagaagatgggcgaccatctgaccaacatccagaggctcgttggctcccaagctgggctgggcgagtatctctttgaaaggctcactctcaagcacgactaagtcgacctgcaggcatgcaagctt